(19) United States
(12) Patent Application Publication
(10) Pub. No.: US 2024/0197980 A1
(43) Pub. Date: Jun. 20, 2024

(54) DEPLOYABLE SPONGE SYSTEM

(71) Applicant: Ezriel Kornel, Bedford, NY (US)

(72) Inventor: Ezriel Kornel, Bedford, NY (US)

(21) Appl. No.: 18/285,776

(22) PCT Filed: Apr. 6, 2022
(86) PCT No.: PCT/US2022/023663

§ 371 Date: Oct. 5, 2023

Related U.S. Application Data
(60) Provisional application No. 63/171,343, filed on Apr. 6, 2021.

(51) Int. Cl. A61M 1/00; A61M 25/00; A61M 27/00; A61M 39/22

(52) U.S. Cl. CPC A61M 1/916; A61M 25/0082; A61M 27/00; A61M 39/22

(57) ABSTRACT

A deployable sponge system comprising: a lumen including a proximal end and a distal end positioned opposite the proximal end; a catheter movably mounted in the lumen, wherein the lumen is flexible and hollow; and a sponge element mounted on a distal end of the catheter and configured to fit in the lumen and movable with the catheter in the lumen from a retracted position in the lumen to an extended position in which the sponge element extends beyond the distal end of the lumen; wherein the catheter includes at least one distal opening formed in the distal end thereof and configured to allow fluid to flow from the sponge element into the catheter.

### CROSS-REFERENCE TO RELATED APPLICATIONS

The present application claims benefit of and priority to U.S. Provisional Patent Application Ser. No. 63/171,343 filed Apr. 6, 2021 and entitled DEPLOYABLE SPONGE SYSTEM, the entire content of which is hereby incorporated by reference herein.

### BACKGROUND

### Field of the Disclosure

The present disclosure relates to a deployable sponge system that includes a deployable and retractable sponge element for use in absorbing fluid from a patient's body.

### Related Art

The use of sponges made of various materials, such as a 4×4 gauze, to absorb blood and other fluids in a patient's body both during surgical procedures and to treat wounds is common. Conventional sponges are typically manually applied in or to the patient's body and left in place in or on the body to absorb fluid. Once the conventional sponge becomes saturated, it is removed manually.

Drainage of fluid from the body and especially from wounds, including infected wounds is important for proper healing. One commonly used device for draining fluid from infected wounds typically includes a sponge connected to suction and placed in the open wound while covered by a plastic adhesive layer. The adhesive layer must be removed every few days and the sponge must be changed, however, the wound is left open to accommodate the sponge and allow for fluid removal. While the wound closes over time, it is essentially left open during the entire healing process to allow access for drainage of fluid which is inconvenient and may also increase the likelihood of additional complications.

In certain surgical procedures, for example spinal surgery, a drain may be left in place after the procedure for a couple of days to drain any blood and other fluids that have collected. The tubes used in these drainage systems may clog which prevents them from functioning properly which may lead to further complications.

Accordingly, it would be desirable to provide a system for removing fluid from a patient's body that avoids these and other problems.

### SUMMARY

In light of the above, a deployable sponge system is disclosed.

The present disclosure is generally related to a deployable sponge system including a sponge element that may be deployed in a user's body and retracted after use.

A deployable sponge system in accordance with an embodiment of the present disclosure includes: a lumen including a proximal end and a distal end positioned opposite the proximal end; a catheter movably mounted in the lumen, wherein the lumen is flexible and hollow; a sponge element mounted on a distal end of the catheter and configured to fit in the lumen and movable with the catheter in the lumen from a retracted position in the lumen to an extended position in which the sponge element extends beyond the distal end of the lumen, wherein the catheter includes at least one distal opening formed in the distal end thereof and configured to allow fluid to flow from the sponge element into the catheter.

In embodiments the deployable sponge system includes a flange formed on the lumen between the proximal end and the distal end and configured to secure the lumen to a body of a patient.

In embodiments, a proximal diameter of the proximal end of the lumen is larger than a distal diameter of the distal end of the lumen.

In embodiments, the lumen may be substantially straight.

In embodiments, the lumen may be bent in at least one direction along its length.

In embodiments, the deployable sponge system may include: a first movable element provided at the distal end of the lumen; and a second movable element provided at the distal end of the lumen, wherein the first movable element is movable relative to the second movable element and the second movable element is movable relative to the first movable element when that the sponge element passes between the first movable element and the second movable element to the extended position.

In embodiments, the lumen may include a valve element positioned at the distal end to open and close the distal end of the lumen to allow the sponge element to extend to the extended position.

In embodiments, the valve element may include: a first movable valve element, and a second movable valve element, wherein the first movable valve element and the second movable valve element are movable relative to each other between a closed position in which the first movable valve element is in contact with the second movable valve element to close the distal end of the lumen and an open position in which the first movable valve element and second movable valve element separate such that the sponge element passes between them to the extended position.

In embodiments, the lumen may include a first static seal positioned in the distal end of the lumen and configured to provide a seal around a proximal portion of the sponge element when the sponge element is in the extended position and to compress the sponge element to better fit in the lumen when the sponge element is retracted into the lumen from the extended position.

In embodiments, the lumen may include a second static seal element positioned in the proximal end of the lumen and configured to compress the sponge element for insertion into the proximal end of the lumen.

In embodiments, the catheter may include a blocker element extending outward from an outer wall of the catheter and having a blocker diameter larger than the proximal diameter of the proximal end of the lumen such that the blocker limits movement of the catheter and lumen.

In embodiments, the blocker element may be positioned on the catheter to set a maximum extended position of the sponge element and the catheter.

In embodiments, the deployable sponge system may include a fastening element configured to releasably secure the blocker element to the catheter such that the position of the blocker element on the catheter is adjustable.

In embodiments, the lumen may include a compressible valve positioned in the distal end of the lumen and configured to selectively close the distal end of the lumen and to selectively open to allow the sponge element and the catheter to pass through into the extended position.

In embodiments, the compressible valve may include: a first compressible element positioned adjacent to the distal opening; and a second compressible element positioned adjacent to the distal opening and in contact with the first compressible element to close the distal end, wherein the first compressible element and the second compressible element are configured to flex to allow the sponge element and the catheter to pass between them and extend to the extended position.

In embodiments, the catheter may include a sponge cone positioned adjacent to a proximal end of the sponge element and configured to seal the distal end of the lumen to prevent fluid from flowing into the lumen when the sponge element and catheter are in the extended position.

In embodiments, the sponge cone may be flexible and flexes to allow the sponge element to retract into the lumen.

In embodiments, the deployable sponge system includes a sponge housing surrounding the sponge element, wherein the sponge housing compresses the sponge element to pass into the proximal end of the lumen.

In embodiments, the sponge housing is configured to pass at least partially into the proximal end of the lumen and the sponge housing separates from the sponge element as the sponge element advances to the extended position.

In embodiments, the catheter includes at least one marking provided on a sidewall thereof indicating a position of the sponge element relative to the lumen.

### BRIEF DESCRIPTION OF THE DRAWINGS

Exemplary embodiments of the present disclosure will be described with reference to the accompanying figures, wherein:

FIGS. 1-1, 1-2 and 1-3 illustrates exemplary schematic views of deployable sponge systems in accordance with embodiments of the present disclosure;

FIG. 1A illustrates the deployable sponge system of FIG. 1-2 with a sponge element thereof in a retracted position in accordance with an embodiment of the present disclosure;

FIG. 1B illustrates the deployable sponge system of FIG. 1-2 with a sponge element thereof in a partially deployed position in accordance with an embodiment of the present disclosure;

FIG. 1C illustrates the deployable sponge system of FIG. 1-2 with a sponge element thereof in a fully deployed position in accordance with an embodiment of the present disclosure;

FIG. 2A illustrates an exemplary schematic view of a deployable sponge system of FIG. 1-1 with a catheter and sponge separated from the lumen in accordance with another embodiment of the present disclosure;

FIG. 2B illustrates the deployable sponge system of FIG. 2A with the sponge element thereof in a retracted position in the lumen in accordance with an embodiment of the present disclosure;

FIG. 2C illustrates the deployable sponge system of FIG. 2A with a sponge element thereof in a deployed position in accordance with an embodiment of the present disclosure;

FIG. 3A illustrates an exemplary schematic view of a deployable sponge system with a sponge element thereof in a retracted position in accordance with another embodiment of the present disclosure;

FIG. 3B illustrates the deployable sponge system of FIG. 3A with the sponge element thereof in a partially deployed position in accordance with an embodiment of the present disclosure;

FIG. 3C illustrates the deployable sponge system of FIG. 3A with the sponge element thereof in a fully deployed position in accordance with an embodiment of the present disclosure;

FIG. 4 illustrates another view of the deployable sponge system of FIGS. 3A-3C in accordance with an embodiment of the present disclosure;

FIG. 5A illustrates a more detailed view of the lumen used in the deployable sponge system of FIG. 4 in accordance with an embodiment of the present disclosure;

FIG. 5B illustrates a cross-sectional view of the lumen of FIG. 5A along the line A-A in accordance with an embodiment of the present disclosure;

FIG. 5C illustrates a proximal end view of the lumen of FIG. 5A;

FIG. 6 illustrates a more detailed view of the lumen used in the deployable sponge system of FIGS. 2A-2C in accordance with an embodiment of the present disclosure;

FIG. 7 illustrates a more detailed view of the lumen and the catheter and sponge element used in the deployable sponge system of FIGS. 2A-2C in accordance with an embodiment of the present disclosure;

FIG. 8 illustrates a more detailed view of a catheter and sponge element suitable for use in any of the deployable sponge systems of FIGS. 1-7 in accordance with an embodiment of the present disclosure;

FIG. 9 illustrates a more detailed view of a catheter and sponge element suitable for use in any of the deployable sponge systems of FIGS. 1-7 in accordance with an embodiment of the present disclosure;

FIG. 10A illustrates an exemplary schematic view of a deployable sponge system with a sponge element thereof removed from the lumen in accordance with another embodiment of the present disclosure;

FIG. 10B illustrates an exemplary schematic view of the deployable sponge system of FIG. 10A with the sponge element thereof in the retracted position in accordance with an embodiment of the present disclosure;

FIG. 10C illustrates an exemplary schematic view of the deployable sponge system of FIG. 10A with a sponge housing thereof removed from the sponge in accordance with an embodiment of the present disclosure;

FIG. 11A illustrates an exemplary schematic view of a deployable sponge system with a sponge element thereof in a retracted position in accordance with an embodiment of the present disclosure;

FIG. 11B illustrates an exemplary schematic view of the deployable sponge system of FIG. 11A with the sponge element thereof in a partially deployed position in accordance with an embodiment of the present disclosure;

FIG. 11C illustrates an exemplary schematic view of the deployable sponge system of FIG. 11A with the sponge element thereof in a fully deployed position in accordance with an embodiment of the present disclosure;

FIG. 12A illustrates an exemplary schematic view of a deployable sponge system with a sponge element thereof in a retracted position in accordance with another embodiment of the present disclosure;

FIG. 12B illustrates an exemplary schematic view of the deployable sponge system of FIG. 12A with a sponge element thereof passing through the lumen thereof I a partially deployed position in accordance with an embodiment of the present disclosure;

FIG. 12C illustrates an exemplary schematic view of the deployable sponge system of FIG. 12A with the sponge element thereof in a fully deployed position in accordance with an embodiment of the present disclosure;

FIG. 13A illustrates an exemplary schematic view of a deployable sponge system with the sponge element thereof in a retracted position in accordance with another embodiment of the present disclosure;

FIG. 13B illustrates an exemplary schematic view of the deployable sponge system of FIG. 13A with the sponge element thereof advanced in the lumen but still in the retracted position in accordance with an embodiment of the present disclosure;

FIG. 13C illustrates an exemplary schematic view of the deployable sponge system of FIG. 13A with the sponge element thereof in a partially deployed position in accordance with an embodiment of the present disclosure;

FIG. 13D illustrates an exemplary schematic view of the deployable sponge system of FIG. 13A with the sponge element thereof advanced in a fully deployed position in accordance with an embodiment of the present disclosure;

FIG. 14A illustrates an exemplary schematic view of a deployable sponge system with a sponge element thereof in a retracted position in accordance with another embodiment of the present disclosure;

FIG. 14B illustrates an exemplary schematic view of the deployable sponge system of FIG. 14A with a sponge element moving through a lumen thereof but still retracted in accordance with an embodiment of the present disclosure;

FIG. 14C illustrates an exemplary schematic view of the deployable sponge system of FIG. 14A with the sponge element thereof in a fully deployed position in accordance with an embodiment of the present disclosure;

FIG. 15 illustrates a cross-sectional view of a lumen used in the deployable sponge system of FIGS. 2A-2C using the valve illustrated in FIGS. 14A-14C in accordance with another embodiment of the present disclosure;

FIG. 16 illustrates a more detailed view of the valve included in the deployable sponge system of FIGS. 14A-14C and the lumen of FIG. 15 in accordance with an embodiment of the present disclosure;

FIG. 17 illustrates a more detailed view of a portion of the valve element included in the deployable sponge system of FIGS. 14A-14C and 15 in accordance with another embodiment of the present disclosure; and

FIG. 18A is a more detailed view of the lumen of FIG. 15 in accordance with an embodiment of the present disclosure; and

FIG. 18B is a cross-sectional view of the lumen of FIG. 18A along the line A-A in accordance with an embodiment of the present disclosure.

### DETAILED DESCRIPTION

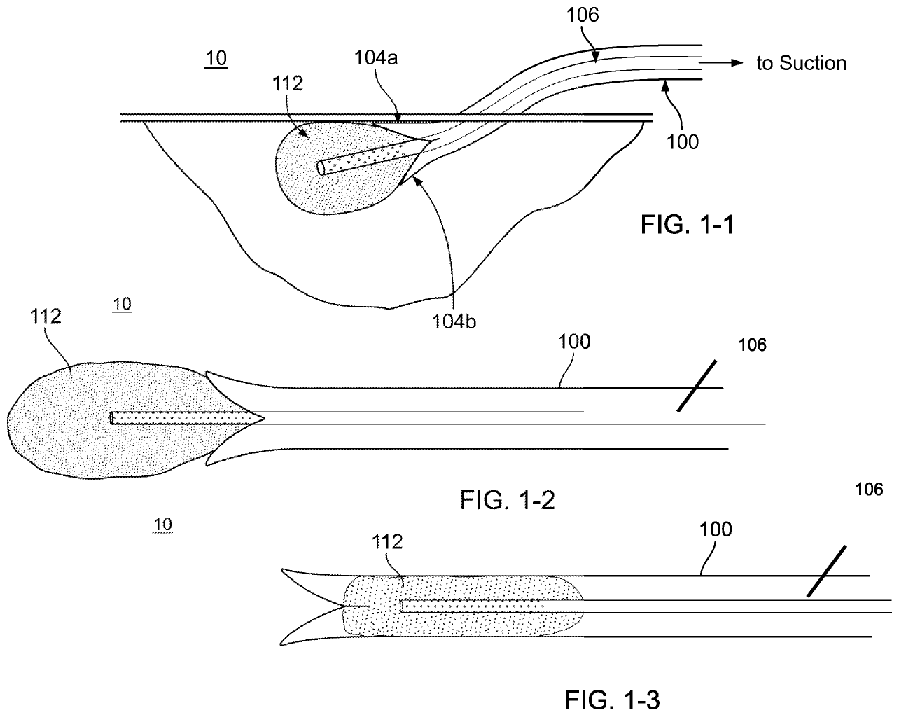

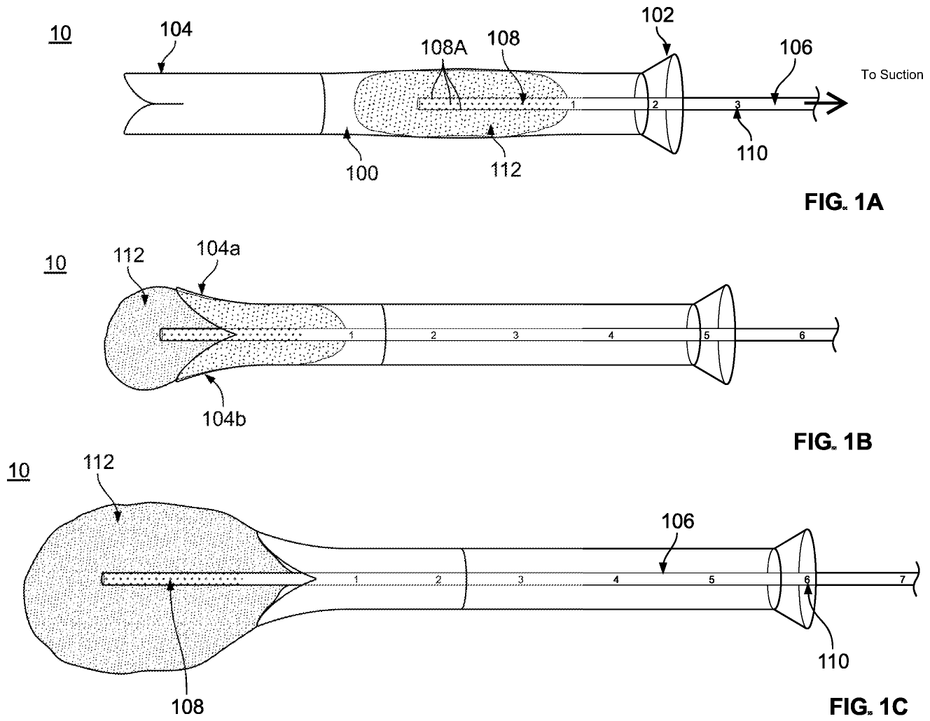

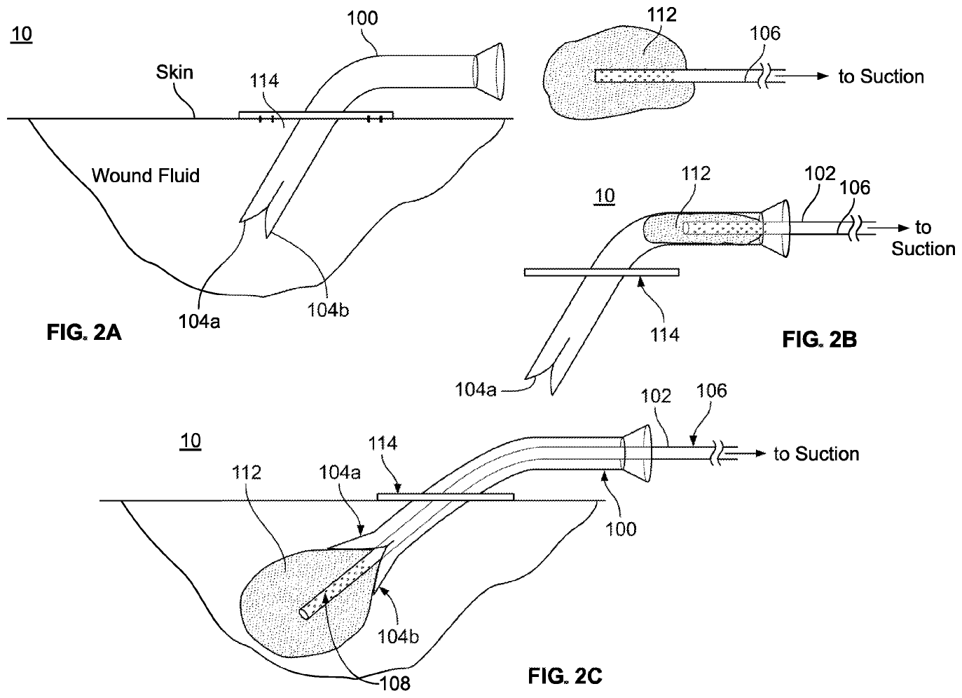

FIGS. 1-1, 1-2 and 1-3 illustrate exemplary embodiments of a deployable sponge system 10 including a lumen 100 with a catheter 106 moveably mounted therein and a sponge element 112 mounted at a distal end of the catheter 106. FIG. 1A shows the deployable sponge system 10 of FIG. 1-2 including the lumen 100, which, in embodiments, may be a hollow tube made of any medically suitable material such as silicone or plastic, to name a few. In embodiments, the lumen 100 may be substantially straight, however, any shape or orientation may be used to allow for clinical placement of the sponge element 112. In embodiments, the lumen 100 may have a proximal end 102 that has a larger diameter than the distal end 104 to allow for easier insertion of sponge element 112 into the lumen 100. In embodiments, the distal end 104 of the lumen 102 may be the end that contacts the patient's body and, in embodiments, has a relatively smaller diameter to allow easier entry into the user's body. In embodiments, the proximal end 102 may not have a larger diameter than the distal end 104. In embodiments, the sponge element 112 may be fixably attached to the catheter 106 such that both the catheter 106 and sponge element 112 are movable in the lumen 100 together. In embodiments, the proximal end of catheter 106 may be attached to a standard suction tube or other suction source (not shown) such that negative pressure may be established in the catheter 106. In embodiments, the catheter 106 may include a distal catheter section 108 (see FIG. 1A, for example) that includes one or more perforations 108a to allow for negative pressure from the standard suction source to be applied through the catheter and to and through the sponge element 112 to draw fluid into the sponge element and through the catheter 106. In embodiments, since fluid is being drawn into the sponge element 112 and then passes into and up the catheter 106, the sponge element generally will not become saturated and may remain in place throughout a procedure.

In embodiments, the catheter 106 may include catheter markings 110 (see FIG. 1A for example) along its length that allow a practitioner to determine how far the catheter 106 and sponge element 112 have been distally deployed in the lumen 100. In embodiments, the catheter markings 110 may be used by the practitioner to set a desired position of the sponge element 112 relative to the distal end 104 of the lumen 100. In embodiments, the catheter 106 may be made of silicone, vinyl, PVC, latex, polyurethane, polyethylene terephthalate (PET) or thermoplastic elastomers, to name a few and may be flexible and malleable. In embodiments, the catheter 106 may be made of any suitable and medically appropriate material.

In embodiments, the sponge element 112 may be any suitable biocompatible sponge, including open cell or closed cell foam made of polyester, rayon, cotton, cellulose, polyvinyl alcohol (PVA), polyurethane, or polyester, to name a few. In embodiments, the sponge element 12 may be made of any suitable and medically appropriate material.

FIG. 1A shows the catheter 106 and sponge 112 in a retracted position where the sponge is entirely inside the lumen 100. FIG. 1B illustrates the catheter 106 as it is pushed distally into and through the lumen 100 such that the sponge element 112 is in a partially deployed or extended position with a portion thereof extending out past the distal end 104 of the lumen 100. In embodiments, the distal end 104 of the lumen 100 includes a first end portion 104a and second end portion 104b that are flexible or movable such that they separate to allow the sponge element 112 to pass therethrough and into or into contact with the patient's body in a deployed or extended position. In embodiments, the first end portion 104a and the second end portion 104b may be made of a flexible and resilient material such that when the sponge element 112 applies pressure, the first end portion 104a and the second end portion 104b flex away from each other and the sponge element extends out between them and when the sponge element is retracted, the first end portion 104a and the second end portion 104b return to the original position in contact with each other.

FIG. 1C illustrates the sponge element 112 in a fully deployed or extended position extending out beyond the distal end 104 of the lumen 100 and the first and second end portions 104a and 104b. In embodiments, the perforations 108a in the distal catheter section 108 allow negative pressure from the suction tube and/or suction source applied to the proximal end of the catheter 106 to pull fluid from the body and into the sponge element 112 and through the catheter 106 when the sponge is in the deployed position in the user's body.

As can be seen in FIG. 2A, for example, in embodiments, the distal end 104 of the lumen 100 may be positioned in a patient's body, under their skin such that the sponge element 112 may absorb fluid which may then be removed from the user's body and the sponge element via the catheter 106. In embodiments, pockets of fluid may be found at wound sites or in the user's body and the system 10 may be used to remove the fluid.

In embodiments, as illustrated in FIGS. 2A-2C, the lumen 100 may have a curved or angled shape. In embodiments, the curved or angled shape of the lumen may bend in at least one direction. In embodiments, the lumen 100 may include a flange 114 that allows the lumen 100 to be attached to the patient's skin via stitches, staples or any other medically acceptable method to hold it in place. In embodiments, the flange 114 may be positioned such that it lays substantially flush on the user's skin while the distal end 104 of the lumen 100 extends into the user's body. As can be seen in FIG. 2C, for example, when the sponge element 112 is in the fully deployed, or extended, position, it extends through the end portions 104a, 104b and into the user's body. As noted above, the negative pressure provided via the catheter 106 may draw liquid from the user's body through the sponge element 112 and into the catheter for removal. FIG. 2A illustrates the sponge element 112 in a retracted position and FIG. 2B shows it in a partially deployed or extended position where a small portion of the sponge element 112 extends beyond the first end portion 104a and the second end portion 104b of the lumen. In the fully deployed position of FIG. 2C substantially all of the sponge element 112 extends beyond the first end portion 104a and the second end portion 104b to maximize surface area exposed to the fluid to be absorbed.

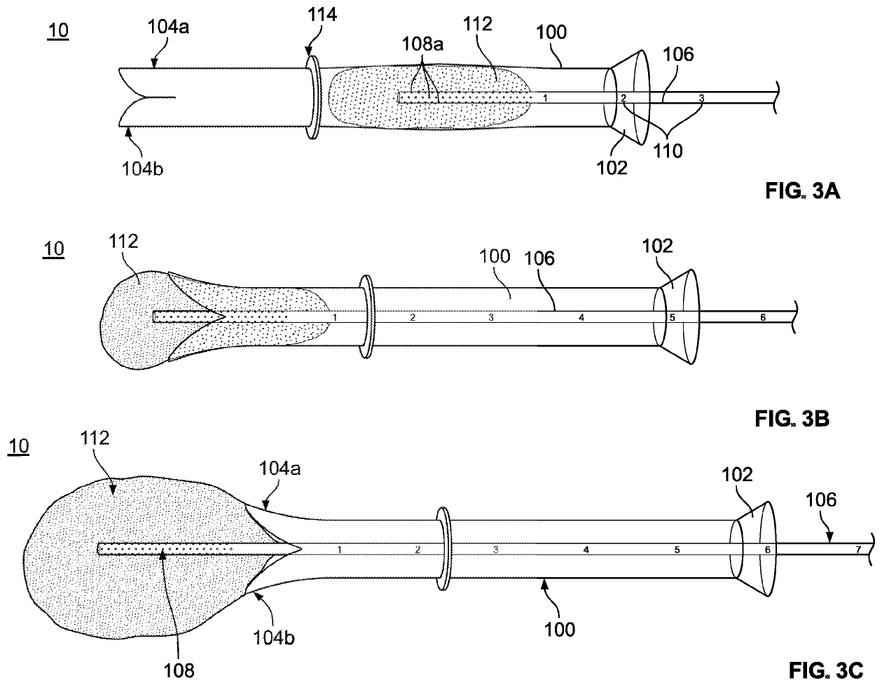

In embodiments, the lumen 100 may be substantially straight in shape as can be seen in FIGS. 1A-1C and 3A-3C, for example. In embodiments, the straight lumen 100 may include the flange 114 which may be configured and operable in a manner similar to the flange 114 discussed above with respect to FIG. 2. The flange 114 is illustrated in FIGS. 3A-3C, where the sponge element 112 is in the retracted position in FIG. 3A, in the partially deployed position in FIG. 3B and in the fully deployed position in FIG. 3C.

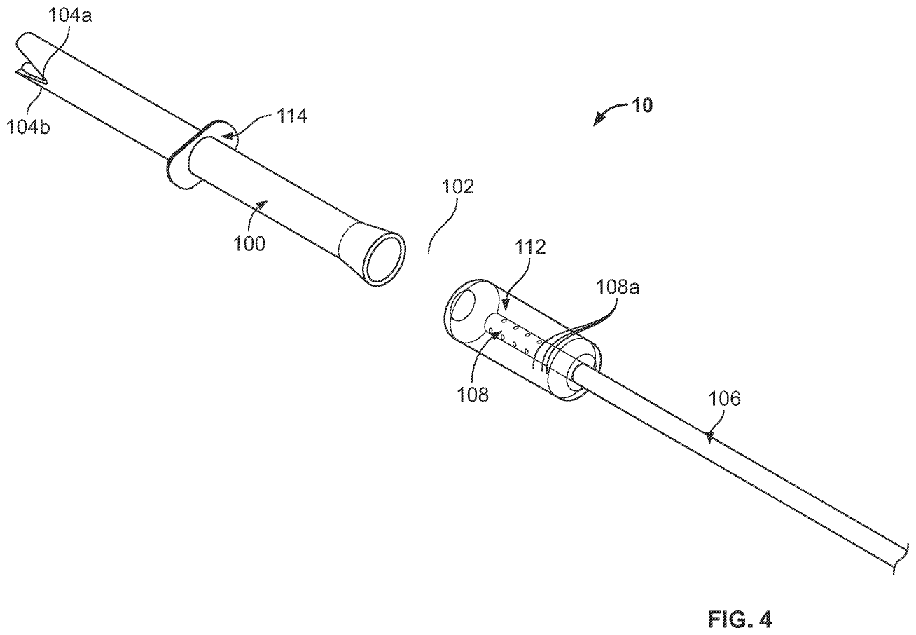

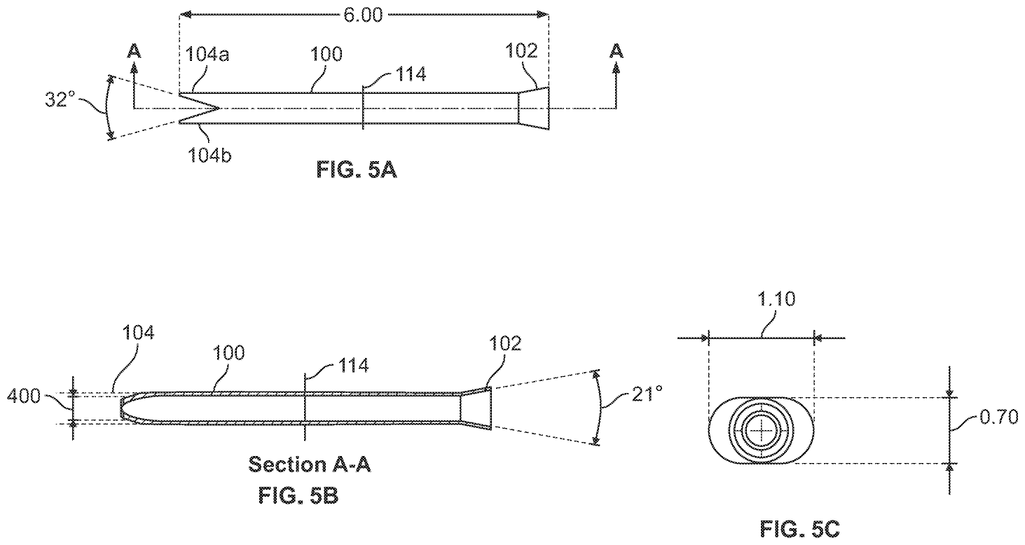

FIG. 4 illustrates the lumen 100 of FIGS. 3A-3C separated from the catheter 106 such that the sponge element 112 is clearly visible. FIG. 5A shows a more detailed view of the lumen 100 of FIGS. 3A-3C and 4 while FIG. 5B shows a cross-sectional view of the lumen of FIG. 5A along the line A-A. FIG. 5C illustrates a proximal end view of the lumen 100 of FIG. 5A. The dimensions illustrated in FIGS. 5A-5C are exemplary and may vary based on application or medical needs.

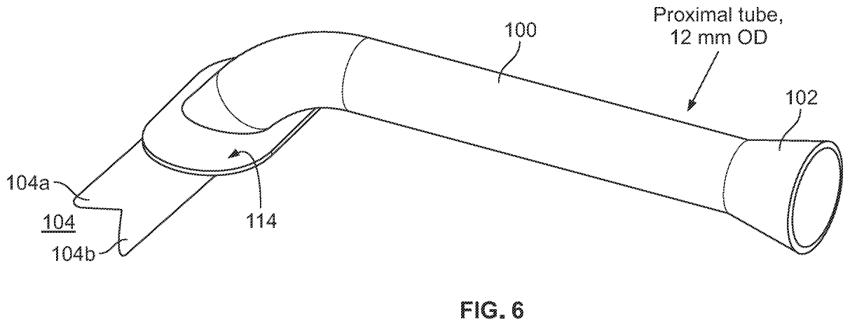

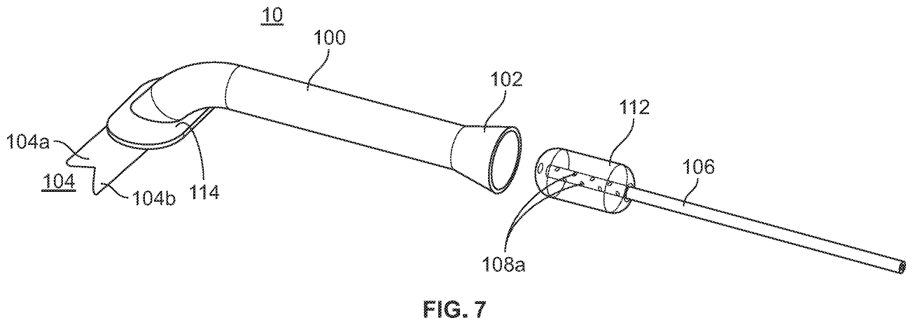

FIG. 6 illustrates a more detailed view of the lumen 100 used in the deployable sponge system of FIGS. 2A-2C. The dimensions illustrated in FIG. 6 are exemplary and may vary depending on application or medical needs. FIG. 7 illustrates a more detailed view of the deployable sponge system of FIGS. 2A-2C with the catheter 106 and sponge element 112 removed from the lumen 100.

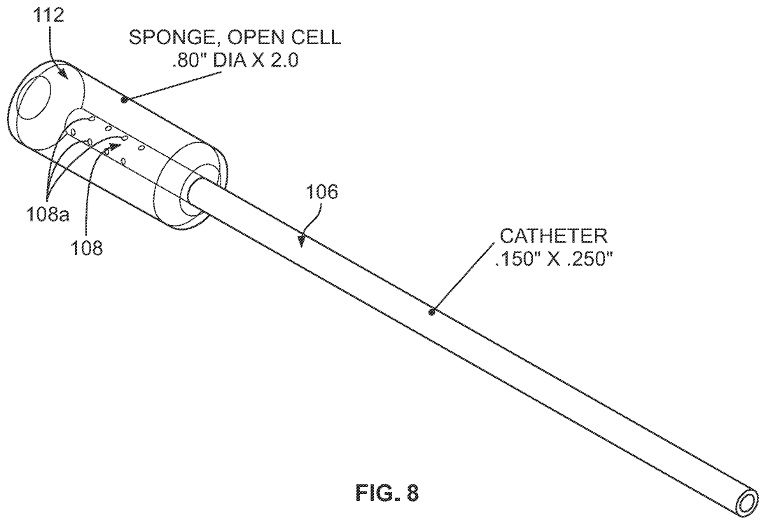

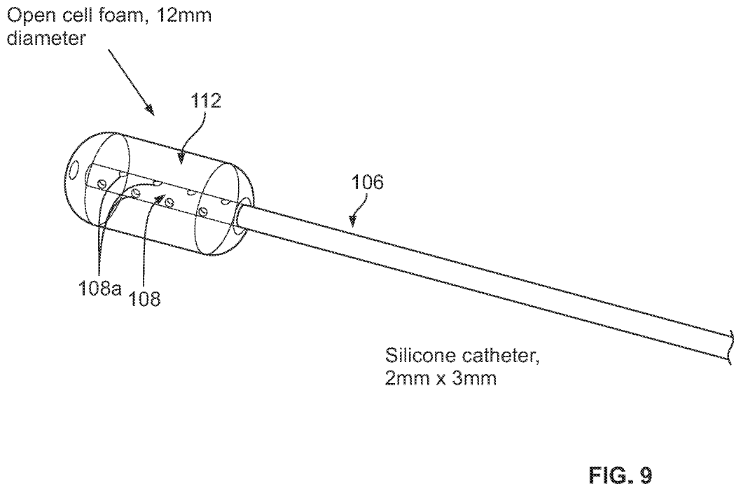

FIG. 8 illustrates a catheter 106 and sponge element 112 suitable for use in any of the deployable sponge system of FIGS. 1-4 and 7. FIG. 9 illustrates another catheter 106 and sponge element 112 suitable for use in any of the deployable sponge system of FIGS. 1-4 and 7. The dimensions indicated in FIGS. 8-9 are exemplary and may vary depending on lumen size, application and/or medical needs.

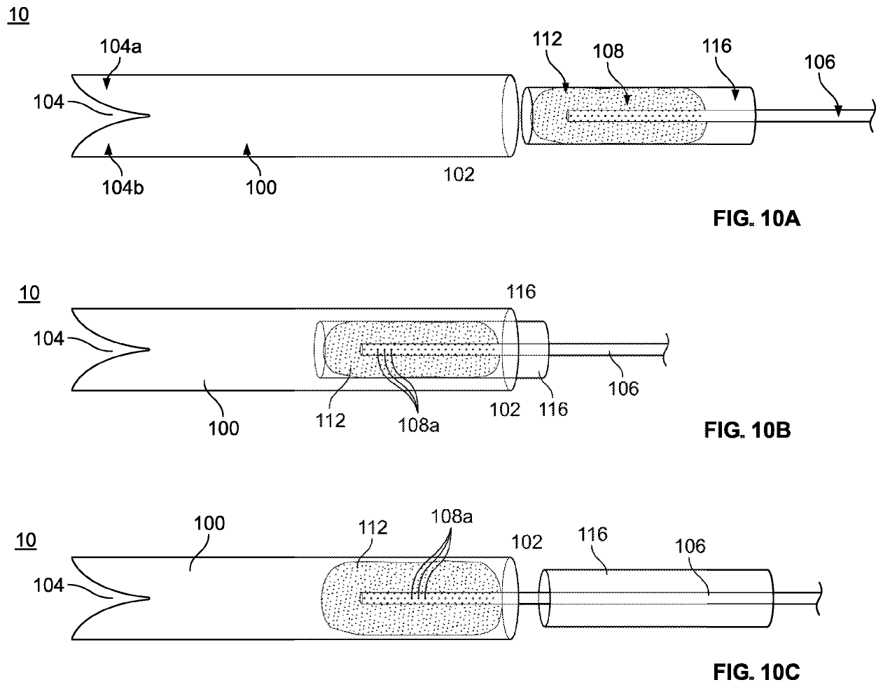

In embodiments, a sponge housing 116 may be provided around the sponge element 112 to pre-compress the sponge element 112 prior to insertion into the proximal end 102 of the lumen 100 as can be seen in FIGS. 10A-10C. FIG. 10A illustrates the sponge element 112 and the catheter 106 separated from the lumen 100 prior to insertion therein. In embodiments, the sponge element 112 may be provided in the sponge housing 116 and the sponge housing may have an external diameter less than the diameter of the lumen 100, or at least the proximal end 102 thereof such that the housing 116 may pass at least partially into the lumen 100 as can be seen in FIG. 10B. In embodiments, after the sponge housing 116 and sponge element 112 are inserted into lumen 100, the sponge housing 116 may be removed as can be seen in FIG. 10C, for example, and the sponge element 112 may be pushed and inserted through the remainder of the lumen 100 and out the end portions 104a, 104b and into the patient's body. In embodiments, the sponge housing 116 may be retracted from the lumen 100 via the proximal end 102 as the catheter 106 and sponge element 112 are advanced forward as can be seen in FIG. 10C, for example. In embodiments, the sponge housing 116 may be removed from the lumen 100 after the sponge element 112 is inserted. In embodiments, the sponge housing 116 may remain in the lumen 100 while the sponge element 112 extends beyond the distal ends 104a, 104b of the lumen. In embodiments, the sponge housing 116 may be flexible.

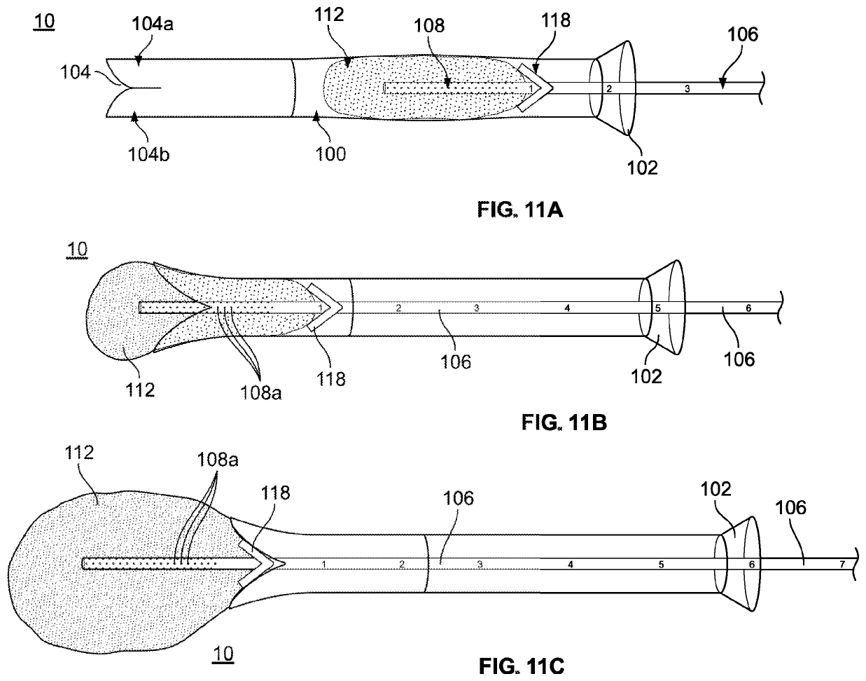

In embodiments, the catheter 106 may include a sponge cone 118 that is fixed on the catheter 106 near the proximal end of sponge 112 as can be seen in FIGS. 11A-C, for example. In embodiments, the sponge cone 118 has a genera cone shape and may be fixed to the catheter and assists in pushing sponge 112 through lumen 100, past the first end portion 104a and the second end portion 104b. In embodiments, the sponge cone 118 may also assist in retraction of sponge 112 back through distal end portions 104a and 104b of the lumen 100. In embodiments, the sponge cone 118 surrounds at least a portion of a proximal end of the sponge element 112 to allow for easier passage through the first end portion 104a and the second end portion 104b of the lumen 100 when the sponge element is being retracted. FIG. 11A shows the sponge element 112 with the sponge cone 118 in a retracted position in the lumen 100. In FIG. 11B, the sponge element 112 is advancing through the first end portion 104a and the second end portion 104b such that the sponge element 112 is partially deployed. In FIG. 11C, the sponge element 112 is in the deployed or extended position and extends beyond first end portion 104a and the second end portion 104b. As illustrated in FIG. 11C, for example, the sponge cone 118 may hold the first end portion 104a and the second end portion 104b in a separated position to allow for easy passage of the sponge element 112 back through the end portions 104a, 104b when it is retracted back into the lumen 100.

In embodiments, the sponge cone 118 may also form a seal with the first end portion 104a and the second end portion 104b to prevent liquid from entering the lumen 100 even when the sponge element 112 is in the deployed or extended position such that all of the liquid passes through the sponge element 112 and the catheter 106 such that it is removed from the body. In embodiments, the sponge cone 118 may not form a seal and fluid may be allowed to flow around it into the lumen 100. In embodiments, the sponge cone 18 may be flexible and resilient.

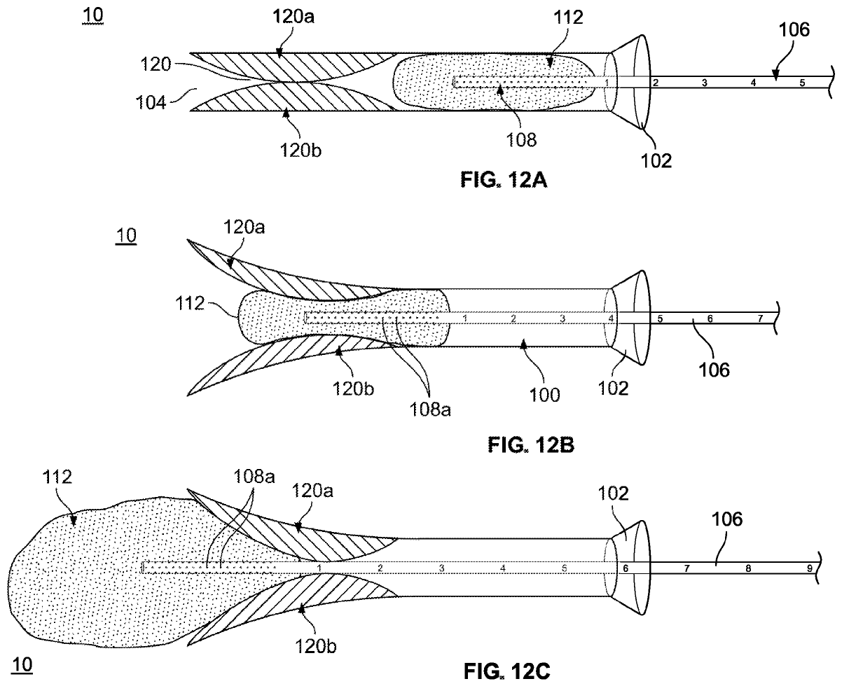

In embodiments, the lumen 100 may include a distal valve 120 including a first valve portion 120a and second valve portion 120b provided at the distal end 104 of the lumen as can be seen in FIGS. 12A-12C, for example. In embodiments, the first valve portion 120a and the second valve portion 120b are movable relative to each other from a closed position where they contact each other and block the distal end 104 of the lumen 100 to prevent fluid from entering, as can be seen in FIG. 12A, for example. In the closed position, the first and second valve portions 120a, 120b provide a seal to prevent fluid from the user's body from entering the lumen 100 other than through the catheter 106 and sponge element 112 when they are extended either partially or fully through the open end 104. In an open position, the first and second valve portions 120a, 120b separate to allow the sponge element 112 to pass therethrough, at least partially as can be seen in FIG. 12B, for example. When the sponge element 112 is in the deployed or extended position shown in FIG. 12C, for example, the first valve portion 120a and the second valve portion 120b provide a seal around the proximal end of the sponge element 112 and the catheter 106 to prevent fluid from entering the lumen 100 other than through the sponge element and catheter to ensure that all fluid is withdrawn. In embodiments, the first valve portion 120a and the second valve portion 120b may be made of a flexible and resilient material to allow them to move between the open and closed positions. In embodiments, the first valve portion 120a and the second valve portion 120b are biased into the closed position.

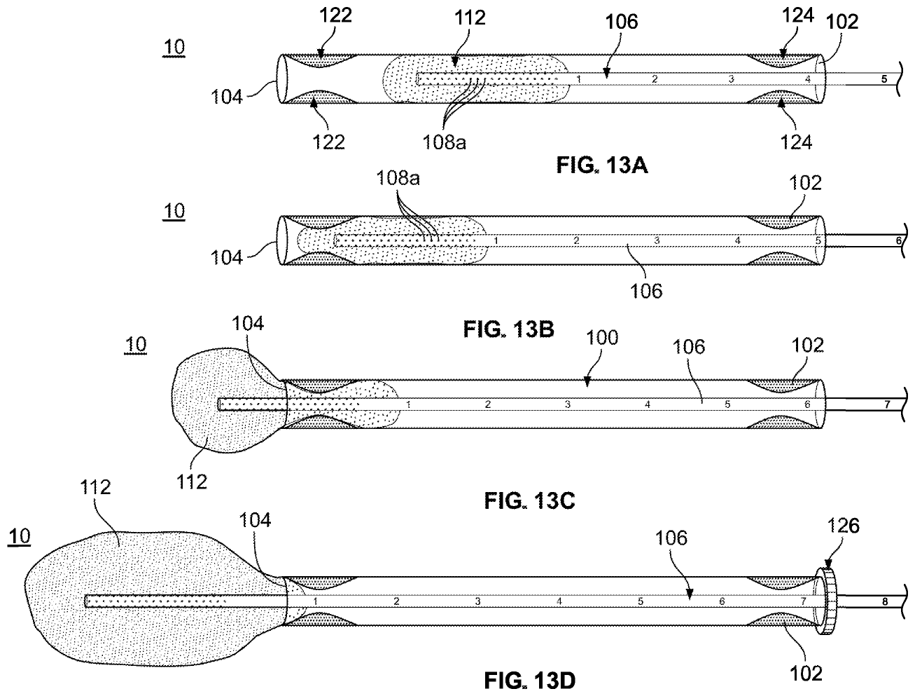

In embodiments, the lumen 100 may include a first static hourglass-shaped seal element 122 positioned at the distal end 104 thereof and a second static hourglass-shaped seal element 124 positioned at a proximal end thereof as can be seen in FIGS. 13A-13D. In embodiments, the first and second hourglass-shaped seal elements 122, 124 are static, that is, they do not move or flex. FIG. 13A illustrates the sponge element 112 and catheter 106 in the retracted position between the first static hourglass-shaped seal 122 and the second static hourglass-shaped seal element 124. In embodiments, the second hourglass-shaped seal element 124 helps initially compress and guide sponge element 112 as it is inserted into the lumen 100 and pushes forward. In embodiments, the first hourglass-shaped seal element 122 also helps to shape and receive the sponge element 112 as it passes forward toward the distal end 104 (see FIG. 13B for example) and when it is retracted back into the lumen 100 after it is extended to the deployed position. As can be seen in FIG. 13C, the first static hourglass-shaped seal element 122 allows the sponge element 112 to pass and expand out through the distal end 104 of the lumen 100. In embodiments, the first hourglass-shaped seal element 122 provides a seal around a proximal end of the sponge element 112 when it is in the extended position, as can be seen in FIG. 13D, for example.

In embodiments, the catheter 106 may include a stopper 126 (see FIG. 13D, for example) that may be fixed thereto and sized such that it will not enter the proximal end 102 of the lumen 100. In this embodiment, the proximal end 102 may or may not have a larger diameter than the remainder of the lumen 100. In embodiments, the stopper 126 allows the clinician to push the exact amount of catheter 106 into the lumen 100 such that sponge element 112 is provided in a desired deployed or extended position. In embodiments, the catheter 106 may include one or more fastening elements configured to secure the stopper 126 thereto. In embodiments, the catheter 106 may include fastening elements at multiple positions along the catheter 106 to allow for adjustment of the stopper position, and thus, adjustment of the extended position of the sponge element 112. In embodiments, the fastening elements may be threaded elements, clips, caps or any other suitable fastening element or structures to secure stopper 126 to the catheter 106 such that the sponge element 112 is provided in a desired deployed or extended position. In embodiments, FIG. 13A shows the sponge element 112 and catheter 106 in the retracted position in the lumen 100. FIG. 13B shows the sponge element beginning to pass through the first seal element 122. FIG. 13C shows the sponge element 112 in a partially deployed position. In FIG. 13D, the sponge element 112 is positioned in the desired deployed or extended position as indicated by the stopper 126. In FIG. 13D, the stopper 126 has a wider diameter than the proximal end 102 of the lumen such that it stops further extension of the catheter 106 and sponge element 112.

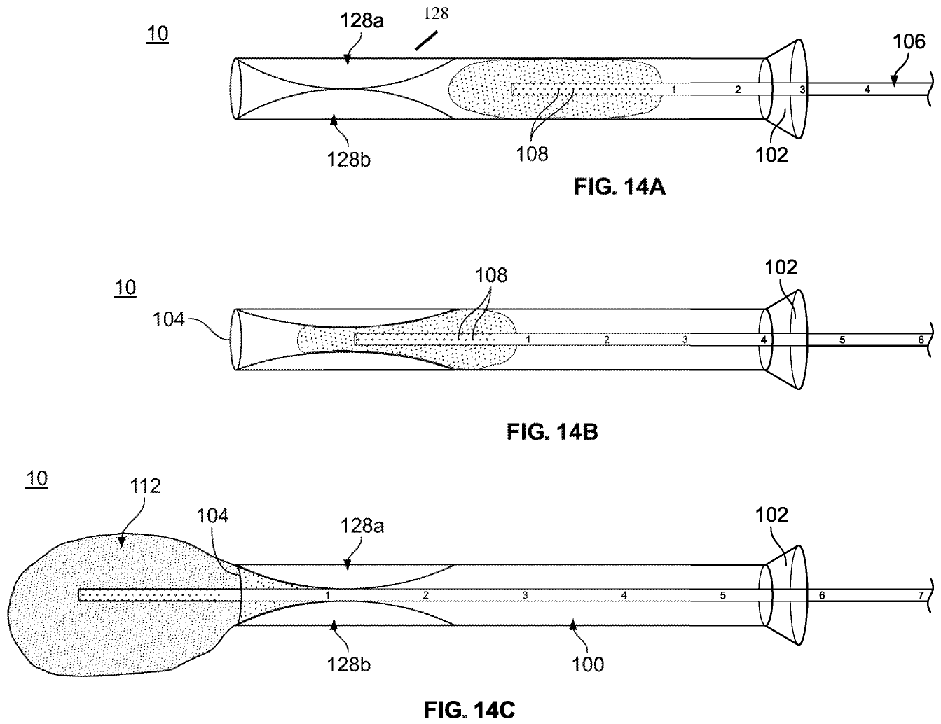

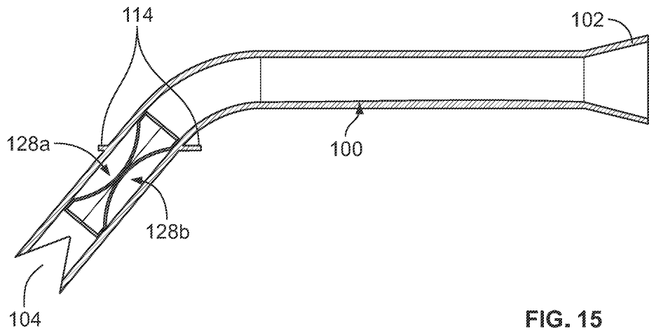

In embodiments, the deployable sponge system 10 may include a lumen 100 with a compressible valve element 128 provided in a distal end 104 thereof as can be seen in FIGS. 14A-14C. In FIG. 14A, the valve assembly 128 is in a fully closed position preventing fluid from entering the lumen 100 from the user's body. In FIG. 14A, the first compressible element 128a and the second compressible element 128b are fully expanded such that they contact each other and create a seal. In FIG. 14B, the sponge element 112 is partially deployed through valve element 128, with the first and second compressible elements 128a, 128b compressing to allow the sponge 112 to pass between them. In FIG. 14C, the sponge element 112 is in the extended position and the first and second compressible elements 128a and 128b expand to form a seal around catheter 106 to prevent fluid from traveling around the outside of the catheter 106 and further into the lumen 100. FIG. 15 illustrates an alternate embodiment of the lumen 100 wherein the lumen has a curved or angled shape similar to that of FIGS. 2A-2C and includes the compressible valve element 128 discussed above with respect to FIGS. 14A-14C positioned at the distal end thereof discussed above with respect to FIGS. 14A-14C.

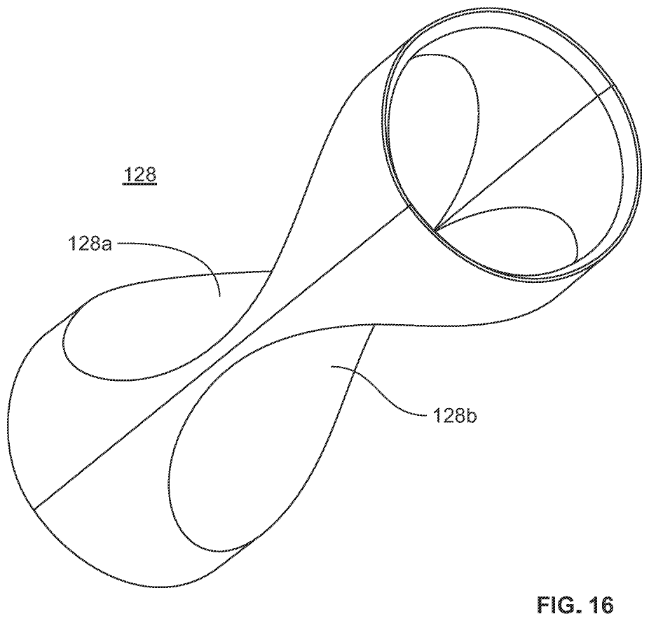

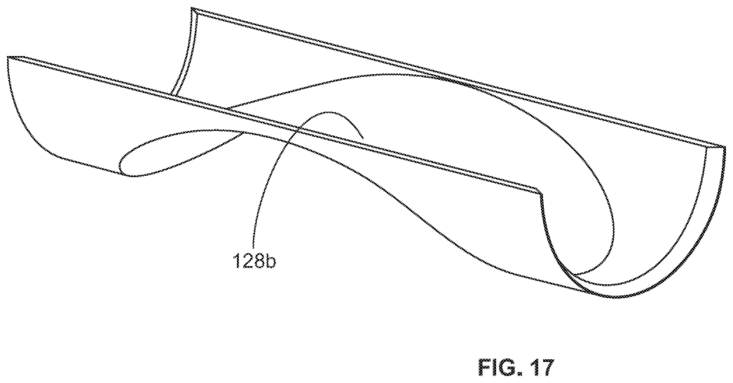

FIG. 16 is a more detailed view of the compressible valve element 128 used in the exemplary embodiments of FIGS. 14A-14C and 15. FIG. 17 is a more detailed view of the second compressible element 128b of the compressible valve element 128 shown in the exemplary embodiments of FIGS. 14A-14C and FIG. 15.

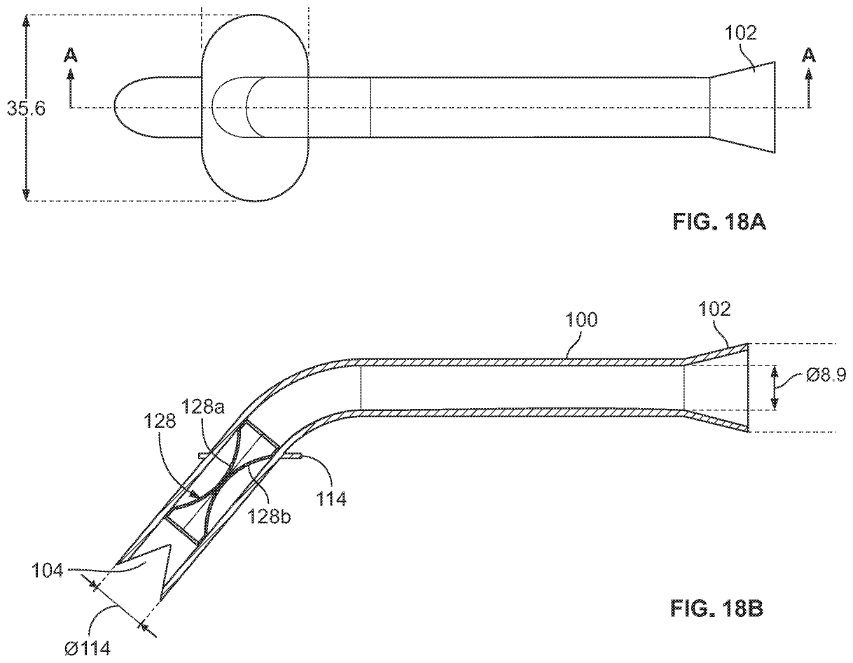

FIG. 18A is a more detailed view of the lumen 100 used in the deployable sponge system 10 of FIGS. 14A-14C and FIG. 18B is a cross-sectional view of the lumen 100 if FIG. 18A along the line A-A. The dimensions included in FIGS. 18A-18B are exemplary and may vary depending on application and medical needs.

In embodiments, the system 10 may provide advantages over conventional sponges and fluid removal system used to treat wounds and in medical procedures. The sponge element 112 may be mounted on and move with the catheter 106 which extends outside the user's body when the sponge 112 is deployed in the patient's body such that the chances that the sponge element 112 will be inadvertently left in the user's body are substantially reduced. Further, since the sponge element 112 is mounted on the catheter 106 which provides suction to draw fluid through the sponge element and into the catheter, the sponge element 112 does not become saturated such that it does not need to be changed, or changed less often, which saves time and reduces the chance of infection. In addition, since the sponge element and catheter 106 are inserted through the lumen 100, which may be inserted under the users skin and secured in place, via the flange 114, for example, such that the system 10 may be left in place, after surgery for example, to provide drainage. Since the fluid is absorbed through the sponge element 112 which has many pores, blockage is unlikely. Further, the lumen 100 may inserted into and remain in place in an infected wound while the wound opening is closed around it such that fluid may be drained without leaving the wound open.

In operation, a practitioner or other medical personnel may make an incision in a user's body in an area in which the sponge element 112 is needed to absorb fluid. In embodiments, where there is a break in the skin already because of an existing or infected wound, no incision need be made. In embodiments, the sponge element 112 may be used to absorb fluid outside the body as well. In embodiments, the lumen 100 may be inserted into the user's body through the incision, or placed in the wound. Where the lumen is positioned in a wound, the interior of the wound may be properly debrided before the skin layer is closed around the lumen such that the wound need not be left open. In embodiments, the catheter 106 and sponge element 112 may be inserted into the proximal end 102 of the lumen 100 and extended through the lumen and into the user's body or wound and into the extended position. In embodiments, the sponge element 112 may be used to absorb fluid which may be drawn into the catheter 106 via the one or more openings 108a and into the catheter 106 and out of the body. In embodiments, the sponge element 112 may be retracted through the lumen 100 and removed from the user's body out the proximal end of the same lumen. In embodiments, another sponge element 112 and catheter 106 may be inserted into the lumen 100 and used to remove fluid. Replacement of the sponge element 112 and/or catheter 106 after a few days or a week may be particularly useful when the system is used to treat a wound. After all fluid is removed, the lumen 100 may be removed from the user's body. In embodiments, the lumen 100 may be left in place for future procedures or removed, if desired.

While particular embodiments of the present disclosure have been shown and described in detail, it would be obvious to those skilled in the art that various modifications and improvements thereon may be made without departing from the spirit and scope of the disclosure.

What is claimed is:

1. A deployable sponge system comprising:
a lumen including a proximal end and a distal end positioned opposite the proximal end;
a catheter movably mounted in the lumen, wherein the lumen is flexible and hollow; and
a sponge element mounted on a distal end of the catheter and configured to fit in the lumen and movable with the catheter in the lumen from a retracted position in the lumen to an extended position in which the sponge element extends beyond the distal end of the lumen;
wherein the catheter includes at least one distal opening formed in the distal end thereof and configured to allow fluid to flow from the sponge element into the catheter.

2. The deployable sponge system of claim 1, further comprising a flange formed on
the lumen between the proximal end and the distal end and configured to secure the lumen to a body of a patient.

3. The deployable sponge system of claim 1, wherein a proximal diameter of the proximal end of the lumen is larger than a distal diameter of the distal end of the lumen.

4. The deployable sponge system of claim 1, wherein the lumen is substantially straight.

5. The deployable sponge system of claim 1, wherein the lumen is bent in at least one direction along its length.

6. The deployable sponge system of claim 1, further comprising:
a first movable element provided at the distal end of the lumen; and
a second movable element provided at the distal end of the lumen;
wherein the first movable element is movable relative to the second movable element and the second movable element is movable relative to the first movable element when that the sponge element passes between the first movable element and the second movable element to the extended position.

7. The deployable sponge system of claim 1, wherein the lumen includes a valve element positioned at the distal end to open and close the distal end of the lumen to allow the sponge element to extend to the extended position.

8. The deployable sponge system of claim 7, wherein the valve element comprises:
a first movable valve element; and
a second movable valve element,
wherein the first movable valve element and the second movable valve element are movable relative to each other between a closed position in which the first movable valve element is in contact with the second movable valve element to close the distal end of the lumen and an open position in which the first movable valve element and second movable valve element separate such that the sponge element passes between them to the extended position.

9. The deployable sponge system of claim 1, wherein the lumen further comprises a
first static seal positioned in the distal end of the lumen and configured to provide a seal around a proximal portion of the sponge element when the sponge element is in the extended position and to compress the sponge element to better fit in the lumen when the sponge element is retracted into the lumen from the extended position.

10. The deployable sponge system of claim 9, wherein the lumen further comprises a
second static seal element positioned in the proximal end of the lumen and configured to compress the sponge element for insertion into the proximal end of the lumen.

11. The deployable sponge system of claim 1, wherein the catheter further comprises
a blocker element extending outward from an outer wall of the catheter and having a blocker diameter larger than the proximal diameter of the proximal end of the lumen such that the blocker element limits movement of the catheter and sponge element into the lumen.

12. The deployable sponge system of claim 11, wherein the blocker element is positioned on the catheter to set a maximum extended position of the sponge element and the catheter.

13. The deployable sponge system of claim 11, further comprising a fastening element configured to releasably secure the blocker element to the catheter such that the position of the blocker element on the catheter is adjustable.

14. The deployable sponge system of claim 1, wherein the lumen further comprises a
compressible valve positioned in the distal end of the lumen and configured to selectively close the distal end of the lumen and to selectively open to allow the sponge element and the catheter to pass through into the extended position.

15. The deployable sponge system of claim 14, wherein the compressible valve further comprises:
a first compressible element positioned adjacent to the distal opening; and
a second compressible element positioned adjacent to the distal opening and in contact with the first compressible element to close the distal end,
wherein the first compressible element and the second compressible element are configured to flex to allow the sponge element and the catheter to pass between them and extend to the extended position.

16. The deployable sponge system of claim 1, wherein the catheter further comprises
a sponge cone positioned adjacent to a proximal portion of the sponge element and configured to seal the proximal end of the lumen to prevent fluid from flowing into the lumen when the sponge element and catheter are in the extended position.

17. The deployable sponge system of claim 16, wherein the sponge cone is flexible
and flexes to allow the sponge element to retract into the lumen.

18. The deployable sponge system of claim 1, further comprising a sponge housing
surrounding the sponge element, wherein the sponge housing compresses the sponge element to pass into the proximal end of the lumen.

19. The deployable sponge system of claim 18, wherein the sponge housing is configured to pass at least partially into the proximal end of the lumen and the sponge housing separates from the sponge element as the sponge element advances to the extended position.

20. The deployable sponge system of claim 1, wherein the catheter further comprises
at least one marking provided on a sidewall thereof indicating a position of the sponge element relative to the lumen.
